(19) United States
(12) Patent Application Publication
(10) Pub. No.: US 2012/0123215 A1
(43) Pub. Date: May 17, 2012

(54) LAPAROSCOPIC TISSUE RETRACTOR

(76) Inventor: Nikolai D. M. Begg, Boston, MA (US)

(21) Appl. No.: 12/944,266

(22) Filed: Nov. 11, 2010

(51) Int. Cl. A61B 1/32

(52) U.S. Cl. 600/204

(57) ABSTRACT

A tissue refractor deforms while passing through an injector tool into an expanded or deployed shape suitable for engaging and retracting tissue during a minimally invasive surgical procedure. The tissue retractor may also deform once again into an undeployed shape for extraction from the surgical site.

### BACKGROUND

In laparoscopic procedures and the like, it is often desirable to move tissue or other foreign objects to retract it from obstructing a surgeon's view. For example, within an abdominal cavity, it may be useful to retract liver tissue for an unobstructed view of, or unobstructed physical access to, the kidney. There remains a need for improved tissue retraction methods and devices suitable for use with minimally invasive surgical procedures.

### SUMMARY

A tissue refractor deforms while passing through an injector tool into an expanded or deployed shape suitable for engaging and retracting tissue during a minimally invasive surgical procedure. The tissue retractor may also deform once again into an undeployed shape for extraction from the surgical site.

### BRIEF DESCRIPTION OF THE FIGURES

The invention and the following detailed description of certain embodiments thereof may be understood by reference to the following figures:

FIG. 1 is an exploded view of a tissue retractor and a delivery system.

FIG. 2 shows a cross-section of a tissue retractor.

FIG. 3 shows a finger and a secondary finger of a tissue retractor with the secondary finger undeployed.

FIG. 4 shows a finger and a secondary finger of a tissue retractor with the secondary finger deployed.

FIG. 5 shows a tissue refractor after deployment from a laparoscopic tool.

FIG. 6 shows the deployment of a needle and suture from a tissue retractor.

FIG. 7 shows tissue secured in a retracted position by a tissue retractor.

FIG. 8 shows a process for retracting tissue in a body cavity.

FIG. 9 is a perspective drawing of a tissue retractor exiting a bore of a tool.

FIG. 10 is a perspective drawing of a tissue retractor exiting a bore of a tool.

FIG. 11 is a perspective drawing of a tissue retractor in a deployed position.

### DETAILED DESCRIPTION

A description is provided herein of a device for retracting tissue within a body cavity along with methods of deploying and using such a device. While the description is provided in the context of laparoscopic procedures, the inventive concepts may equally be applied to endoscopic, arthroscopic, or other types of surgical procedures. Still more generally, the principles of the invention may be usefully applied in any environment where a gripping device is passed through a narrow-bore tool or the like and expanded into a deployed configuration by plastic deformation of the device as it exits a delivery system.

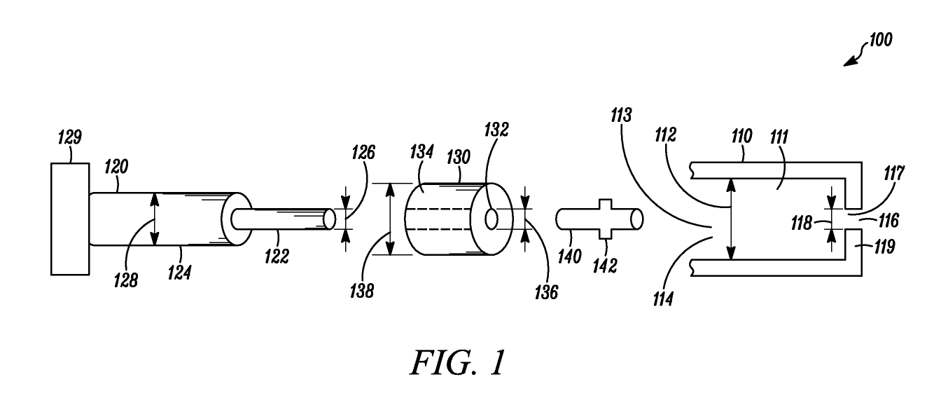

FIG. 1 is an exploded view of a tissue retractor delivery system. The system 100 may include an injector 110, a plunger 120, a sleeve 130, and a device 140 that is deployed to perform tissue retraction, all of which may be aligned when assembled along a common axis passing there through. In general, the injector 110 provides a passage into a body cavity or other working volume, the plunger 120 applies force to inject the device 140 into the body cavity, and the sleeve 130 aligns the device 140 to pass through an exit port 116 of the injector 110. The components of the system 100 are now described in greater detail.

The injector 110 may be shaped and sized to fit within a tool port of a laparoscope (not shown) or the like that delivers the device 140 into a working volume. In general, the injector 110 may include an interior 111 having a first diameter 112. A plunger opening 114 on a first end 113 of the interior 111 may be provided to receive the plunger 120 or similar instrument. An exit port 116 on a second end 117 of the interior 111 opposed to the plunger opening 114 may be shaped and sized to pass a device from the interior 111 into the working volume. The exit port 116 may have a second diameter 118 less than the first diameter 112 of the interior 111.

A flange 119 or other protrusion may be provided at the exit port 116 to narrow the interior 111 of the injector 110 from the first diameter 112 to a smaller diameter such as the second diameter 118 of the exit port 116. More generally, the flange 119 may present a change in dimensions between the interior 111 and the exit port 116 that can plastically deform a device passing there through into a deployed position as described in greater detail below. Depending upon the shape of the device, the flange 119 may present a shelf or step, or some more gradual taper such as a parabolic taper, arced taper, linear taper, and so forth. The flange 119 may also or instead taper to a cross-sectional shape smaller than the second diameter 118 of the exit port 116, and then increase in size to the second diameter 118. Alternatively, the flange 119 may be a shelf, a linear transition, or a curved taper.

The flange 119 may extend around the entire exit port 116, or may include one or more protrusions or the like that narrow the diameter or otherwise extend into the interior 111 only in specific locations or areas around a circumference of the exit port 116. Thus while a transition from the larger interior 111 to the smaller exit port 116 is depicted as a right angle providing the flange 119, it will be understood that the flange 119 may be formed from any shape(s) or dimension(s) suitable to plastically deform a device as described below. More generally, it should be understood that the plastic deformation contemplated herein may be effected through a variety of physical configurations, and all such configurations, shapes, tapers, and so forth that might be recognized by one of ordinary skill as useful for plastically deforming a passing device may be adapted for use with the systems and methods disclosed herein, and all such variations are intended to fall within the meaning of the term “flange” as used herein.

The interior 111 may be a cylindrical interior such as commonly found in conventional laparoscopic tools; however, it will be understood that the interior 111 may also or instead include a cross-sectional shape that is hexagonal, octagonal, square, triangular, oval, or any other suitable shape for delivering devices such as those described herein into a body cavity. Further, alignment grooves, ribs, constrictions, or other structures may be used to hold a device or portion of a device in a desired position within the laparoscopic tool or to prevent a device from rotating during insertion or movement within the interior 111. Thus while this description refers generally to a “diameter,” this is not intended to imply a specific cross-sectional shape (e.g., circular) except to the extent that the exit port 116 narrows this shape in a manner that can deform an object passing from the interior 113 through the exit port 116.

The plunger opening 114 may be shaped and sized to receive a plunger or similar device that is used to drive the device 140 through the interior 111 by an application of force. The injector 110 may be a trocar or other laparoscopic tool. The injector 110 may be inserted into an incision or other opening in a body on which surgery is being performed. The plunger 120 may include a narrower portion 122 having a third diameter 126 less than the second diameter 118 and a wider portion 124 having a fourth diameter 128 greater than the second diameter 118 and less than the first diameter 112. The plunger 120 may be tapered from the fourth diameter 128 to the third diameter 126. The system 100 may further comprise a gripping device 129 attached to the plunger 120 such as a handle or other handgrip. The plunger 120 may be attached to other devices to aid in the holding or manipulation of the plunger 120 or other parts of the system 100. The wider portion 124 may also include various apparatus for securing the plunger 120 in a given location wherein the apparatus may include a screw, a bolt, a wing nut, or similar sort of a securing device. The plunger 120 may be used to push the device 140 through the injector 110 so that the device 140 may be inserted into a body on which surgery is being performed.

The sleeve 130 may have a second interior 132 with a fifth diameter 136 greater than the third diameter 126 and an exterior 134 with a sixth diameter 138 less than the first diameter 112. The sleeve 130 thus dimensioned may be interposed between the plunger 120 and the device 140 so that the sleeve 130 holds the device 140 in a position along an axis of the injector 110 so that the device 140 is aligned with, and may be pushed through, the exit port 116 of the injector 110.

The device 140 may have a cross-sectional shape with a diameter less than the second diameter 118 with at least one protrusion 142 extending beyond the second diameter 118 when the device 140 is placed within the injector 110. The protrusion 142 may be a tab or the like that contacts one or more fingers within the device 140. The device 140 may be deployed through the exit port 116 of the injector 110 by placing the device 140 into the sleeve 130, placing the sleeve 130 into the interior 111 of the injector 110, and inserting the plunger 120 into the injector 110 to drive the sleeve 130 and the device 140 toward the exit port 116. The device 140 may also include a number of fingers as described below that plastically extend from the device 140 when the device 140 is deployed through the exit port 116. Once the device 140 is inserted into a working volume such as a body cavity, the fingers may then be used for tissue retraction. The fingers may form an expandable rake or other suitable configuration for engaging and retracting tissue under a surgeon or other user's control. The device 140 may also include a number of secondary fingers, each connected to a respective one of the fingers by an elastic hinge that permits the secondary finger to expand away from the finger into an open deployment when engaged with tissue or the like while urging the secondary finger back into a closed deployment in the absence of external forces.

The device 140 and the sleeve 130 may be disposable after use. The plunger 120 and the injector 110 may be autoclavable after use so that they may be sterilized and reused in further surgeries. The injector 110, the plunger 120, the sleeve 130, and the device 140 may all be manufactured from biocompatible material. The injector 110 and the plunger 120 may each be formed from stainless steel. Springs, bushings, or washers may be used in between the various components of the system 100 in order to aid in holding one or more of the components in position in preparation of deployment of the device 140. More generally, the components of the system 100 may be adapted in a variety of ways for use as a reusable (e.g., sterilizable) or disposable surgical device, or for other applications.

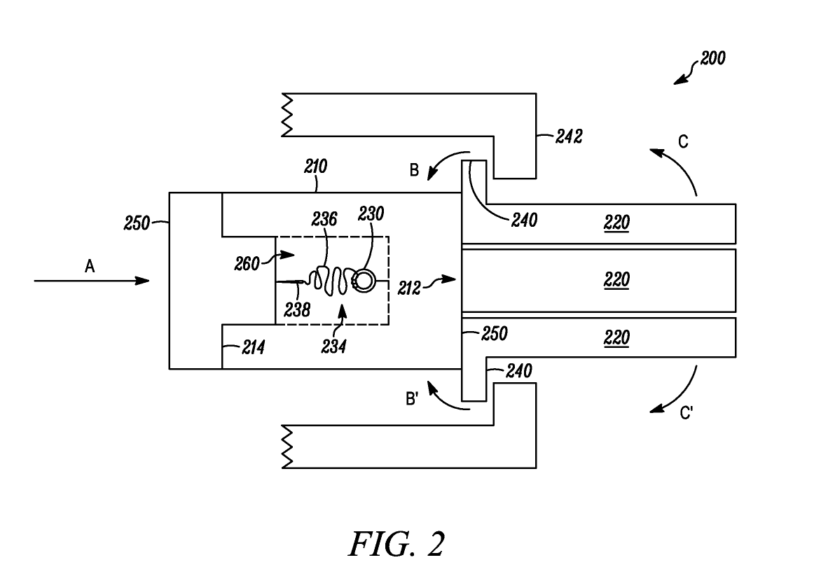

FIG. 2 shows a cross-section of a tissue retractor in an undeployed state. The device 200, which may be any of the devices described above, may include a base 210, a plurality of fingers 220, and a connection point 230.

In general, the base 210 provides a structural anchor for the plurality of fingers 220 and the connection point 230. The base 210 may have a cross section shaped to pass through a bore of a laparoscopic tool such as the injector described above. The base 210 may, for example, have a generally cylindrical form with a circular cross section, or any other shape suitable for passing through the injector. The base 210 may include a first end 212 coupled to the plurality of fingers 220 and a second end 214 opposing the first end 212. A cavity 234 may be provided in the second end 214 (and opening toward the second end 214). More generally, the base 210 may have any shape and size suitable for the purposes described herein, and may include additional fingers, protrusions, or the like to which the fingers 220 are attached.

The connection point 230 may be provided to accommodate an application of force on the device 200 so that the device 200 can be positioned within a body cavity or the like during a procedure. The device 200 may include a suture 236 attached to the connection point 230, and may further include a needle 238 attached to the suture 236. In one aspect, the connection point 230 includes an eyelet for attaching the suture 232. The term “suture” as used herein is intended to include any surgical thread, silk, wire, gut, or other natural or synthetic threaded material that might be used to apply force to the connection point 230 in its deployed state. In one aspect, the device 140 may be sized to fit within a 12 mm diameter laparoscopic trocar or may alternatively be sized to fit within a 5 mm diameter laparoscopic trocar, or any other commercially available size such as 3 mm, 7 mm, 8 mm, 10 mm, 11 mm, 13 mm, or 15 mm, or still more generally, any size suitable for minimally invasive surgery.

The device 200 may generally be any device that might be usefully deployed within a body cavity, such as any of the tissue retractor devices described in greater detail below. The device 200 may include one or more tabs 240, which may be any protrusions that contact a flange 242 of an exit port when the device 200 moves through the exit port. During this movement (indicated by an arrow A), the flange 242 can apply a force to the tabs 240 (indicated by arrows B & B′) that causes a deformation of the fingers 220 into a deployed position, as indicated by arrows C & C′.

The fingers 220 may be coupled to the base 210 with a hinge 250 such as a plastic hinge or the like so that the fingers 220 can plastically deform during ejection into an open rake configuration where the fingers 220 are spread apart from each other. It will be understood that while the deformation may include plastic deformation into the deployed position, this deformation may also be static or elastic deformation. Thus, for example, the fingers 220 may be elastically collapsed to fit into a tool for deployment such that the fingers 220 return to the deployed configuration in the absence of external forces (e.g., when the device 200 has been ejected from the tool). Alternatively, the fingers 220 may be maintained in the deployed configuration by a mechanical support, latching mechanism, guide or control wire, or any other suitable mechanism that can retain the deployed configuration against elastic forces that collapse the device 200 in the absence of external forces. In other embodiments, each hinge 250 may statically or frictionally maintain a current position. This may be achieved, for example, through a tight, frictional engagement between components of a barrel hinge, pivot hinge, or other two-part hinge, or through a detent mechanism or the like that securely maintains the hinge in a number of discrete positions across a range of motion. Thus, the hinge 250 may generally maintain a deployed shape through operation of plastic deformation, elastic deformation, or static deformation, all as generally described above.

The fingers 220 may include three fingers as depicted in FIG. 2, although more generally any number of fingers 220 might be suitably employed, such as two fingers 220, four fingers 220, or some other number. The fingers 220 may also be capable of elastically or plastically deforming from the open rake configuration into a closed configuration when the device 200 is drawn back through the exit port to extract the device 200 from a body cavity or other environment. The connection point 230 may be used to facilitate this process by providing a location on the base 210 at which force can be applied to extract the device 200. It will be understood that while the hinge 250 is depicted simply as a location where a finger 220 couples to the base 210, the hinge 212 may have any shape, and be formed of any material, suitable for the properties and functions described herein, e.g., plastic deformation into a deployed position and plastic or elastic deformation back to an undeployed position. Thus in one aspect the fingers 220 and base 210 may be a single, integral structure formed of plastic with a living hinge (such as a narrowing of the material that joins the fingers 220 to the base 210) or the like therebetween, or the hinge 212 may be a separate structure with springs, resilient materials, and/or plastically deformable materials, or any combination of the foregoing suitable for plastic and/or elastic deformation as contemplated herein.

At the same time, it will be understood that the hinges may be capable of elastic or plastic deformation from an open rake configuration back into a closed configuration that can fit back through an exit port in order to retract the device 200 from a cavity into which the device 200 was deployed.

The base 210 may include a cavity 234 for storing the suture 232 and/or a needle 238, and an opening 260 for accessing the suture 232 and/or needle 238. An end cap 250 may be provided to cover the opening 260 of the cavity 234. The end cap 250 may be shaped and sized to removably cover the opening 236 to enclose the cavity 234 during handling and the like. The end cap 250 may for example be fitted to the opening 260 with a friction fit, or the end cap 250 may be threaded or otherwise shaped to engage corresponding surfaces of the opening 260. In one aspect, the end cap 250 may be attached to a wall of the cavity 234 with a suture, hinge, or the like in order to maintain a physical coupling of the end cap 250 to the base 210 so that they can more readily be removed together from a body cavity or the like. The end cap 250 may also or instead include a separate attachment point so that the end cap 250 can be secured and removed from the body cavity independently from the base 210. The needle 238 may be press fit or otherwise secured to the end cap 250 (as illustrate) or elsewhere within the cavity 234 in order to prevent knotting of the suture 236 during handling and deployment of the device 200.

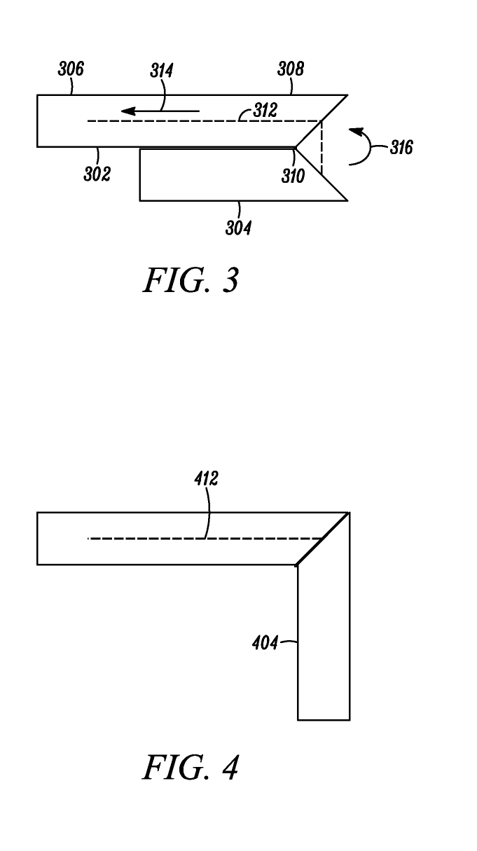

FIG. 3 shows a finger and a secondary finger of a tissue retractor with the secondary finger undeployed. In general, a finger 302 of a tissue retractor may include a secondary finger 304 that can be further expanded to engage tissue for retraction. The finger 302 may be coupled to a base (not shown), which may be any of the bases described above, on a first end 306. A second end 308 of the finger 302 may be hingeably coupled to the secondary finger 304 through a hinge 310, which may be formed in the same manner as any of the hinges described above, or using any other suitable technique. In one aspect, the hinge 310 may be an elastic hinge that urges the secondary finger 304 to open away from the finger 302 in the absence of external forces. In this manner, the secondary finger 304 can open into a deployed position to engage tissue as soon as the tissue retractor is deployed into a working volume such as a body cavity. In another aspect, deployment of the secondary finger 304 may be controlled. For example, the hinge 310 may be an elastic hinge that urges the secondary finger 304 into a closed position parallel to the finger 302, and a suture 312 or the like may be provided to permit external control of deployment by an application of axial force (indicated by a horizontal arrow 314) to create a rotational force about the hinge 310 (illustrated by a curved arrow 316).

It will be understood that the second finger 304 may also or instead be formed from a terminal portion of the finger 302. Thus, for example, the finger may include a bendable or hooked piece of metal formed in an end thereof that serves as the secondary finger 304. In addition the finger 302 may plastically deform into a bent, secondary portion using techniques similar to those described above, or the finger 302 may include a shape memory alloy (such as such as Nickel Titanium alloys) or other shape memory material, which may include one-way or two-way memory for controllable deployment of secondary extensions for gripping tissue or the like. In general, shape memory alloys and other shape memory materials may respond to temperature, electrical current, magnetism, or any other energy by returning to a remembered shape. As used in the devices described herein, the remembered shape may generally include any deployed or undeployed shape for fingers and or secondary fingers of a tissue retractor or similar device. Thus for example each secondary finger may be formed in whole or in part from a shape memory alloy.

FIG. 4 shows a finger and a secondary finger of a tissue retractor with the secondary finger deployed. In general, the finger and the secondary finger may be any of the fingers and secondary fingers described above. Once a tissue retractor is deployed in a body cavity or the like, the secondary finger may be further deployed into a position at which the secondary finger can engage tissue for retraction, as illustrated in FIG. 4. The secondary finger 404 may be retained in the deployed position with tension on the suture 412, or the secondary finger 404 may plastically deform to remain in the deployed position, or the secondary finger 404 may spring into the deployed position when an external retainer (such as the interior wall(s) of an injector) is removed, all as generally discussed above. While the suture 412 is one way to apply a biasing force to a hinge between a finger and a secondary finger, it will be understood that any other arrangement suitably for applying a biasing force about the hinge may also or instead be employed. In another aspect, the hinge for the secondary finger 404 may rotate freely over some range of motion so that the finger can be rotated into an orientation where the secondary finger 404 deploys (or alternatively, undeploys) under the force of gravity.

The secondary finger 404 may include a hook or other edge or shape suitable for engaging, e.g., soft tissue or other material to be retracted with the tissue retractor.

It will be understood that a variety of variations are possible for the fingers and secondary fingers described above. In general, deployment of the fingers and/or the secondary fingers may be effected by plastic deformation or elastic deformation of hinges, and/or may be controlled externally such as with a suture as described above. In addition, a device may include any number of fingers, and each finger may include any number of secondary fingers, as well as various combinations of the foregoing. All such variations are intended to fall within the scope of this disclosure.

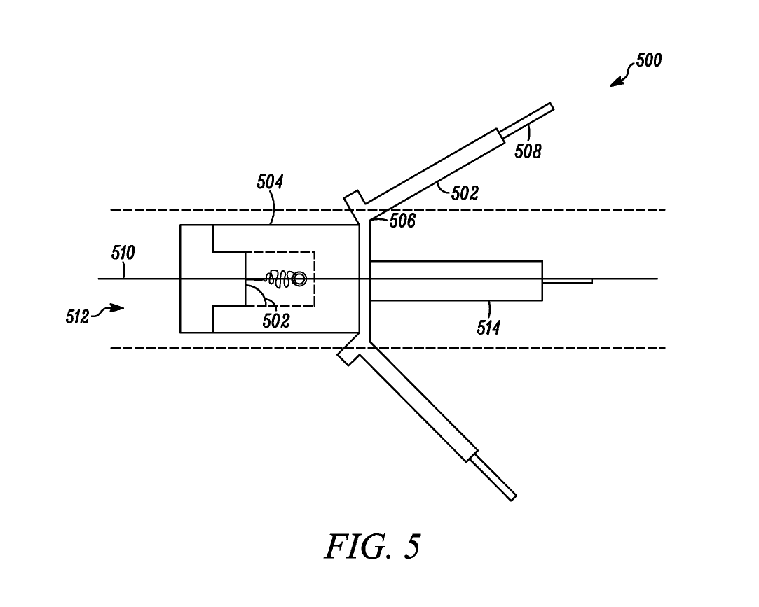

FIG. 5 shows a tissue refractor after deployment from a laparoscopic tool. When deployed, the device 500 may have a number of fingers 502 coupled to a base 504 through hinges 506 or the like that have plastically deformed into an open configuration, all as generally discussed above. A secondary finger 508 for each finger 502 may elastically extend into an open position to engage tissue for retraction. A connection point, suture, and needle may be retained within a cavity of the base 504 until the end cap is removed for access thereto.

The base 504 may generally have an axis 510 passing there through and a cross section normal to the axis 510. The cross section may be shaped to pass through a bore of a laparoscopic tool as discussed above, and generally defines a volume 512 along the axis 510 that encloses the base 504. The tabs that cause plastic deformation may generally be any protrusion that extends beyond the volume such that that they engage a flange or the like as the device 500 passes through an exit port as described above. In one aspect, the finger 502 may move from a first position within the volume 512 (as depicted in FIG. 2) to a second position, e.g., the deployed position, outside the volume 512 as depicted in FIG. 5.

The device 500 may include one or more stationary fingers 514 fixedly coupled to the base 504 so that they do not move relative to the base 504 during the deployment. Nonetheless, all of the fingers 502, stationary fingers 514, and secondary fingers 508 may have a concurrent undeployed position within the volume 512, with the stationary fingers 514 remaining within the volume 512 after deployment. In one aspect, all of the secondary fingers 508 may open in a common direction to engage tissue. Thus, for example, where the plane in which FIG. 5 is drawn represents a plane that divides the base 504 along the axis 510, all of the secondary fingers 508 may open on a common side of the plane. In another aspect, a tether 520 may be provided that secures the end cap to the base 504 independent of the needle/suture assembly. The fingers 502 and secondary fingers 508 may have edges that are rounded or otherwise smoothed to avoid tissue damage during use of the device 500. As depicted, the fingers 502 have a length greater than the secondary fingers 508 to which they are connected, although variations are possible and the secondary fingers 508 may be the same length as the fingers 502, or longer than the fingers 502, all without departing from the scope of this disclosure.

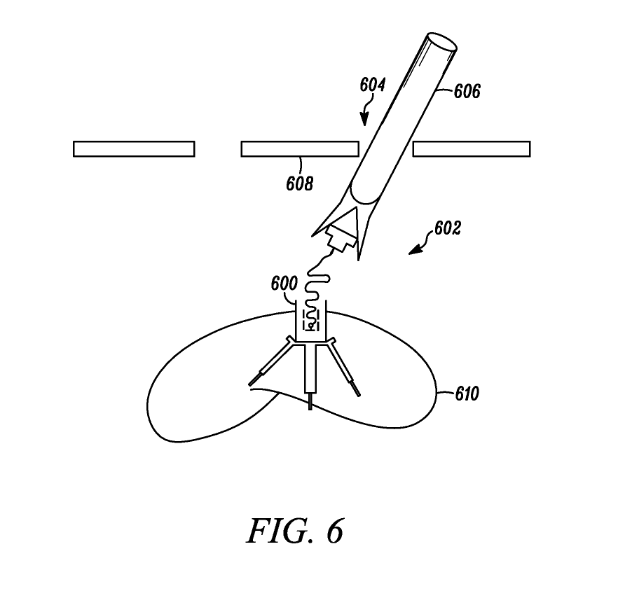

FIG. 6 shows the deployment of the needle and suture from the tissue retractor. After deployment of the device 600 into a working volume 602 via an opening 604 (using, e.g., the injector described above), the device 600 can be manipulated with a tool 606. The working volume 602 may, for example, be a body cavity such as an abdominal cavity, or any other working volume where the device 600 might be usefully deployed. Although only a portion of a wall 608 of the cavity 602 is depicted, it will be generally understood that the cavity 602 may generally be a closed or substantially closed volume have any interior shape, and may be distended with gas or otherwise shaped or manipulated during a surgical procedure as contemplated herein.

The opening 604 may be a laparoscopic incision or other opening through which tools can be inserted and removed, such as a trocar incision for the injector described above. As depicted, more than one opening may be used in a procedure, such as one opening for the tool 606 and another opening for an injector or other tool. In addition, multiple tools may be inserted through multiple openings concurrently according to the complexity and physical reach of a procedure being performed. The tool 606 may be a laparoscopic grasping tool or any other tool useful to position, deploy, and/or manipulate the device 600 or perform a related surgical procedure. In one aspect, the tool 606 may be used to remove an end cap so that a needle and suture can be withdrawn from a cavity of the device 600, all as generally described above. The tool 606 may also be used to position the device 600 to engage tissue 610 (which may be any tissue within a body cavity) for retraction.

One or more additional tools may also be used so that, for example, one tool is used to position the device 600 while another tool is used to grasp the suture, and still another tool is used for a related surgical procedure, and so forth.

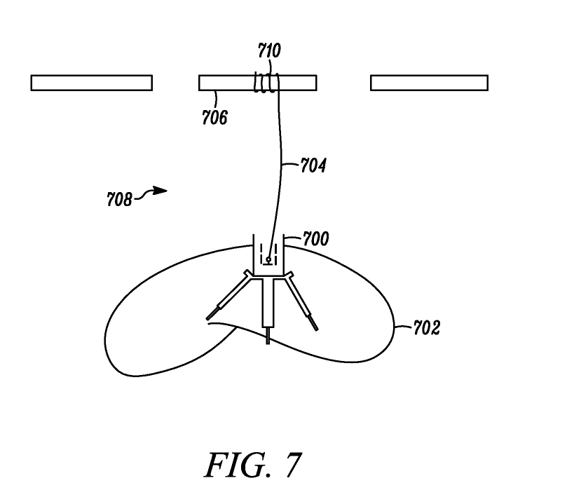

FIG. 7 shows tissue secured in a retracted position by a tissue retractor. Once the device 700 is positioned to engage tissue 702, a suture 704 may be pulled to apply force to the device 700 and retract the tissue 702 in a desired direction. The suture 704 may be secured to a wall 706 of a cavity 708 with a knot 710 or the like, as depicted in FIG. 7. The suture 704 may also or instead be threaded through the wall 706 to an external location where a surgeon or assistant can control the application of force (and thus the amount of retraction) throughout the course of a procedure. All such variations as would be apparent to one of ordinary skill in the art are intended to fall within the scope of this disclosure.

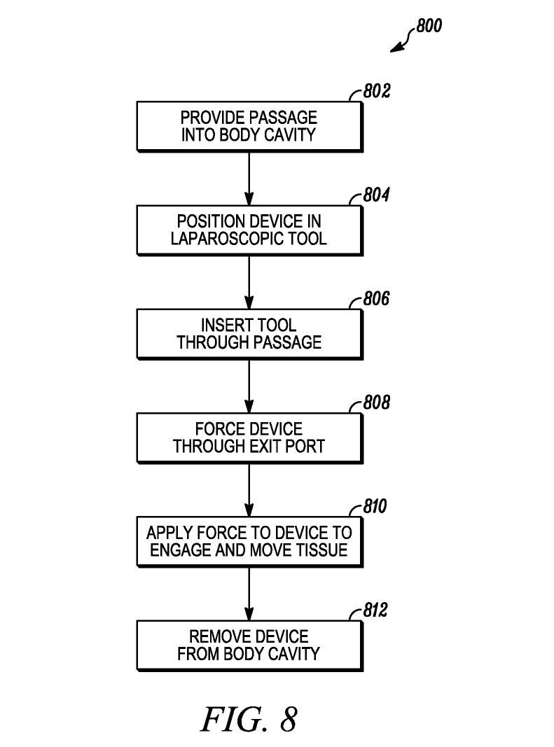

FIG. 8 shows a process for retracting tissue in a body cavity. The process 800 may begin by providing a passage into the body cavity as shown in step 802. This may include, for example making one or more incisions as described above.

As shown in step 804, the process 800 may include positioning a device such as the tissue retractor described above into a laparoscopic tool. The laparoscopic tool may be the injector described above, and positioning may include positioning the device within the tool using a sleeve as described above so that the device is aligned with the exit port.

As shown in step 806, the process 800 may include inserting the laparoscopic tool through the passage created in step 802 and into the body cavity.

As shown in step 808, the process 800 may include forcing the device through an exit port of the laparoscopic tool and into the body cavity in a manner that plastically deforms the device into an expanded rake configuration, or any other deployed position as generally described above. This may, for example, include automatically (e.g., by spring action) or manually (e.g., by pulling a control thread or suture) opening a secondary finger to engage and/or move the tissue.

As shown in step 810, the process 800 may include applying a force to the device to engage and move tissue within the body cavity. This may include, for example, positioning the device to engage tissue, and pulling on the suture to apply a force that retracts the tissue in the direction of pulling, as depicted for example in FIG. 7.

As shown in step 812, the process may include removing the device from the body cavity, such as by once again deforming the device into an undeployed shape (which may be different from an initial undeployed shape) for extraction from a surgical site. In general, the device may be designed to withdraw through the exit port, e.g., by recollapsing or by further deforming into a cross-sectional shape smaller than the exit port, or the device may be withdrawn through the opening or another opening using, e.g., the suture or a grasping tool.

It will be understood that numerous variations to the process 800 described above are possible, and that the steps may be modified, removed, supplemented, and/or re-ordered. All such variations that would be apparent to one of ordinary skill in the art are intended to fall within the scope of this disclosure.

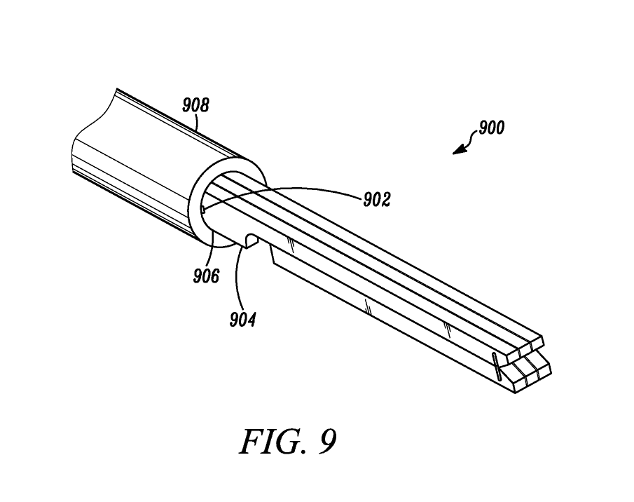

FIG. 9 is a perspective drawing of a tissue retractor exiting a bore of a tool. In general, the tissue retractor 900 may be as described above. In the depicted embodiment, a tab 902 on a finger 904 engages a flange 906 of an exit port as the tissue retractor 900 exits a tool 908 such as a laparoscopic tool.

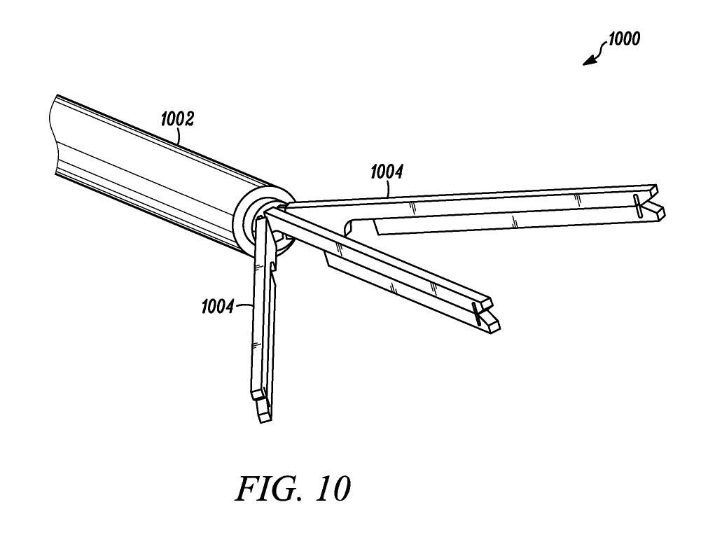

FIG. 10 is a perspective drawing of a tissue retractor exiting a bore of a tool. The tissue retractor 1000 may be any of the tissue retractors described above. As a tab engages a flange of a tool 1002, one or more fingers 1004 of the tissue retractor 1000 may be forced apart into a deployed configuration. The deployed configuration may be maintained by plastic deformation, elastic deformation, or static deformation of hinges that connect the fingers 1004 to a base of the tissue refractor 1000, all as generally described above.

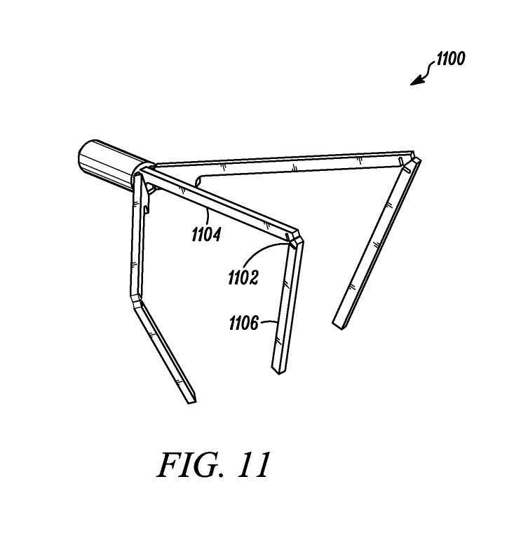

FIG. 11 is a perspective drawing of a tissue retractor in a deployed position. The tissue retractor 1100, which may be any of the tissue retractors described above, may include a hinge 1102 that couples a finger 1104 to a secondary finger 1106. The hinge 1102 may elastically deform into an open position, such as when the secondary finger 1106 engages tissue for retraction. The hinge 1102 may be formed from a piece of elastic material connected between the finger 1104 and the secondary finger 1106 as shown, or more generally of any material or combination of materials suitable for the uses described herein. It will be understood that while three fingers, hinges, and secondary fingers are shown, any number of these components may be usefully employed in a tissue retractor without departing from the scope of this disclosure. The actual number of fingers 1104 in an embodiment of the tissue retractor 1100 may depend, for example, on the size, shape, and resilience of tissue that is being moved, the force to be applied, sensitivity of the tissue to damage, and any number of other factors.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

1. A device comprising:
a base having an axis and a cross section normal to the axis, the cross section shaped to pass through a bore of a laparoscopic tool and the cross section defining a volume about the axis;
a plurality of fingers each having an undeployed position within the volume, one of the plurality of fingers coupled to the base through a hinge capable of a deformation between a first position wherein the one of the plurality of fingers is within the volume and a second position wherein the one of the plurality of fingers is outside the volume;
a tab attached to the one of the plurality of fingers, the tab extending outside of the volume and positioned to apply a force to deform the hinge as the device exits the laparoscopic tool; and
a connection point on the base that permits an application of a force to the base.

2. The device of claim 1 wherein the plurality of fingers form an expandable rake.

3. The device of claim 1 wherein the deformation is a plastic deformation that retains the second position after the deformation.

4. The device of claim 1 wherein the deformation is an elastic deformation that returns to the first position after the deformation.

5. The device of claim 1 wherein the deformation is a static deformation from the first position to the second position, wherein either position is maintained by a frictional engagement with the hinge between the base and the one of the plurality of fingers.

6. The device of claim 1 further comprising a plurality of secondary fingers, each one of the plurality of secondary fingers connected to a respective one of the plurality of fingers by an elastic hinge.

7. The device of claim 6 wherein all of the plurality of fingers and all of the plurality of secondary fingers have a concurrent undeployed position within the volume.

8. The device of claim 6 wherein a first length of one of the plurality of fingers is greater than a second length of the one of the plurality of secondary fingers connected thereto.

9. The device of claim 6 wherein the base is divided by a plane containing the axis, the plurality of secondary fingers each capable of elastically hinging to open onto a common side of the plane.

10. The device of claim 1 further comprising a plurality of secondary fingers, each one of the plurality of secondary fingers formed of a shape memory alloy extending from a respective one of the plurality of fingers.

11-22. (canceled)

23. A device comprising:
a base having a cross section shaped to pass through a bore of a laparoscopic tool;
a plurality of fingers coupled to the base that plastically deform into an open rake configuration when the device exits the laparoscopic tool; and
a connection point on the base that permits an application of a force to the base.

24. The device of claim 23 wherein the plurality of fingers are capable of deforming from the open rake configuration into a closed configuration when the device is drawn through an opening by one of the plurality of fingers.

25. The device of claim 23 wherein the plurality of fingers are capable of deforming from the open rake configuration into a closed configuration when the device is drawn through an opening by a hinge between one of the plurality of fingers and a secondary finger extending from one of the plurality of fingers.

26. The device of claim 23 further comprising a plurality of secondary fingers, each one of the plurality of secondary fingers connected to a respective one of the plurality of fingers by an elastic hinge.

27. The device of claim 23 wherein the connection point includes a suture affixed to the base.

28. The device of claim 27 wherein the base includes a cavity with an opening for storing the suture and a needle.

29. The device of claim 28 further comprising an end cap for covering the opening of the cavity.

30. The device of claim 23 wherein at least one of the plurality of fingers is fixedly coupled to the base.

31. A system comprising:
an injector shaped and sized to fit within a tool port of a laparoscope, the injector including a cylindrical interior having a first diameter, a plunger opening on a first end of the cylindrical interior, an exit port on a second end of the cylindrical interior having a second diameter less than the first diameter, and a flange that narrows the cylindrical interior of the injector from the first diameter to the second diameter;
a plunger including a narrower cylindrical portion having a third diameter less than the second diameter and a wider cylindrical portion having a fourth diameter greater than the second diameter and less than the first diameter;
a sleeve having a second cylindrical interior with a fifth diameter greater than the third diameter and a cylindrical exterior with a sixth diameter less than the first diameter; and
a device having a cross-sectional shape with a diameter less than the second diameter with at least one protrusion extending beyond the second diameter when the device is placed within the injector, wherein the device can be deployed through the exit port of the injector by placing the device into the sleeve, placing the sleeve into the cylindrical interior of the injector, and inserting the plunger into the injector to drive the sleeve and the device toward the exit port.

32. The system of claim 31 further comprising a plurality of fingers that plastically extend from the device when the device is deployed through the exit port.

33-52. (canceled)
